(19) United States
(12) Patent Application Publication
(10) Pub. No.: US 2022/0236803 A1
(43) Pub. Date: Jul. 28, 2022

(54) ACTUATOR DEVICE, METHOD FOR GENERATING VOLTAGE WAVEFORM, METHOD FOR DRIVING FIELD-RESPONSIVENESS POLYMER ACTUATOR, AND PROGRAM

(71) Applicant: TOYODA GOSEI CO., LTD., Kiyosu-shi, Aichi-ken (JP)

(72) Inventors: Keita SUGIYAMA, Kiyosu-shi, Aichi-ken (JP); Takehiko KANZAKI, Kiyosu-shi, Aichi-ken (JP); Ryusuke HORIBE, Kiyosu-shi, Aichi-ken (JP); Kazumasa BABA, Kiyosu-shi, Aichi-ken (JP)

(21) Appl. No.: 17/613,598

(22) PCT Filed: May 26, 2020
(86) PCT No.: PCT/JP2020/020745

§ 371 Date: Nov. 23, 2021

(30) Foreign Application Priority Data
Jun. 19, 2019 (JP) 2019-113712

(51) Int. Cl. G06F 3/01; G06F 3/0484; H01L 41/09

(52) U.S. Cl. CPC G06F 3/016; H01L 41/09; G06F 3/0484

(57) ABSTRACT

An actuator device includes an electroactive polymer actuator that includes two electrodes, a drive unit, and a waveform editing section. The drive unit is configured to drive the electroactive polymer actuator by repeatedly applying, to a section between the two electrodes, a voltage that changes in correspondence with drive waveform data that indicates voltage changes corresponding, to one cycle The waveform editing section is configured to change edit waveform data in correspondence with an operation performed by a user When the edit waveform data is changed during driving of the electroactive polymer actuator, the actuator device is configured to update the drive waveform data such that the changed edit waveform data becomes new drive waveform data and drive the electroactive polymer actuator using the updated drive waveform data.

### TECHNICAL FIELD

The present disclosure relates to an actuator device, a method for generating a voltage waveform, a method for driving an electroactive polymer actuator, and a program.

### BACKGROUND ART

Patent Document 1 discloses a tactile sense presentation device that causes a user to recognize, as a tactile sense, an operation (such as vibration) that is based on the expansion and contraction of an electroactive polymer actuator. The tactile sense presentation device changes the waveform of a voltage applied to the electroactive polymer actuator so as to change operation patterns of the electroactive polymer actuator, thereby presenting the user with various tactile senses that correspond to operation patterns.

### PRIOR ART DOCUMENTS

### Patent Documents

Patent Document 1: Japanese Laid-Open Patent Publication No. 2014-510346

### SUMMARY OF THE INVENTION

### Problems that the Invention is to Solve

To present the user with a particular tactile sense using the tactile sense presentation device, a voltage waveform that operates the electroactive polymer actuator first needs to be generated such that the particular tactile sense is presented. The voltage waveform is generated by, for example, preparing a basic voltage waveform, repeating an editing task that changes the voltage waveform and an operation test of the electroactive polymer actuator using the edited voltage waveform, and causing the tactile sense presented from the electroactive polymer actuator to become close to the particular tactile sense.

It is an object of the present disclosure to improve the efficiency of generating a voltage waveform that produces a particular motion of an electroactive polymer actuator.

### Means for Solving the Problem

An actuator device that solves the above-described problem includes: an electroactive polymer actuator that includes two electrodes; a drive unit configured to drive the electroactive polymer actuator by repeatedly applying, to a section between the two electrodes, a voltage that changes in correspondence with drive waveform data that indicates voltage changes corresponding to one cycle; and a waveform editing section configured to change edit waveform data in correspondence with an operation performed by a user. When the edit waveform data is changed during driving of the electroactive polymer actuator, the actuator device is configured to update the drive waveform data such that the changed edit waveform data becomes new drive waveform data and drive the electroactive polymer actuator using the updated drive waveform data.

In the configuration, when the edit waveform data is changed during driving of the electroactive polymer actuator, the operation of the electroactive polymer actuator is switched to an operation that is based on the changed edit waveform data without performing an operation such as saving or sending the changed edit waveform data. This allows the user to smoothly search for a voltage waveform that produces a particular motion of the electroactive polymer actuator while editing the edit waveform data. As a result, the voltage waveform is generated efficiently.

It is preferred that the actuator device include a change determination section configured to regularly determine whether the edit waveform data has been changed and the actuator device be configured to update the drive waveform data when the change determination section determines that the edit waveform data has been changed

The configuration reduces the frequency of updating the drive waveform data used to drive the electroactive polymer actuator.

It is preferred that the actuator device include an image processor configured to cause a display to display an image that corresponds to the edit waveform data and that the waveform editing section be configured to change the edit waveform data through an operation performed by the user, the operation changing the image displayed on the display using a pointing device.

The configuration allows the user to edit a waveform intuitively. Thus, even a user who has a small amount of knowledge related to machine or information processing easily generates a voltage waveform that produces a particular motion of the electroactive polymer actuator.

It is preferred that the actuator device be employed as a tactile sense presentation device that causes the user to recognize, as a tactile sense, an operation that is based on expansion and contraction of the electroactive polymer actuator.

In the actuator device, it is preferred that the tactile sense presentation device be a pulsation generating apparatus that causes the user to recognize, as a tactile sense of pulsation, vibration that is based on the expansion and contraction of the electroactive polymer actuator.

In the actuator device, it is preferred that when an operation is performed to invoke voltage waveform data stored in advance during driving of the electroactive polymer actuator, the actuator device be configured to change the edit waveform data to the voltage waveform data.

The configuration easily changes the operation of the electroactive polymer actuator during driving to an operation that is based on the voltage waveform data stored in advance.

A method for generating a voltage waveform that solves the above-described problem includes producing a waveform of applied voltage that produces a particular motion of the electroactive polymer actuator using the actuator device.

A method for driving an electroactive polymer actuator that solves the above-described problem includes the steps of: driving the electroactive polymer actuator by repeatedly applying a voltage that changes in correspondence with drive waveform data that indicates voltage changes corresponding to one cycle; changing edit waveform data in correspondence with an operation performed by a user; and when the edit waveform data is changed during driving of the electroactive polymer actuator, updating the drive waveform data such that the changed edit waveform data becomes new drive waveform data. The driving includes driving the electroactive polymer actuator using the drive waveform data updated in the updating.

A program that controls an actuator device that solves the above-described problem includes: an electroactive polymer actuator that includes two electrodes; a drive unit configured to drive the electroactive polymer actuator by repeatedly applying, to a section between the two electrodes, a voltage that changes in correspondence with drive waveform data that indicates voltage changes corresponding to one cycle; and a waveform editing section configured to change edit waveform data in correspondence with an operation performed by a user. When the edit waveform data is changed during driving of the electroactive polymer actuator, the program causes the actuator device to execute a process that updates the drive waveform data such that the changed edit waveform data becomes new drive waveform data and drives the electroactive polymer actuator using the updated drive waveform data.

### Effects of the Invention

The present disclosure improves the efficiency of generating a voltage waveform that produces a particular motion of an electroactive polymer actuator.

### BRIEF DESCRIPTION OF THE DRAWINGS

FIG. 1 is a schematic diagram showing a pulsation generating apparatus.

FIG. 2 is a cross-sectional view showing a dummy.

FIG. 3 is a cross-sectional view showing the cross-sectional structure of a dielectric elastomer actuator.

FIG. 4 is a block diagram showing the pulsation generating apparatus.

FIG. 5 is a diagram illustrating waveform editing screens.

FIG. 6 is a diagram illustrating the waveform editing screens during operation.

FIG. 7 is a flowchart showing the control executed by the waveform editing device while the dielectric elastomer actuator is driven.

FIG. 8 is a flowchart showing the control executed by the drive unit while the dielectric elastomer actuator is driven.

### MODES FOR CARRYING OUT THE INVENTION

An embodiment in which an actuator device of the present disclosure applied to a pulsation generating apparatus will now be described. The pulsation generating apparatus causes a user to recognize, as a tactile sense of the pulsation of a human body, the vibration generated in correspondence with applied voltage.

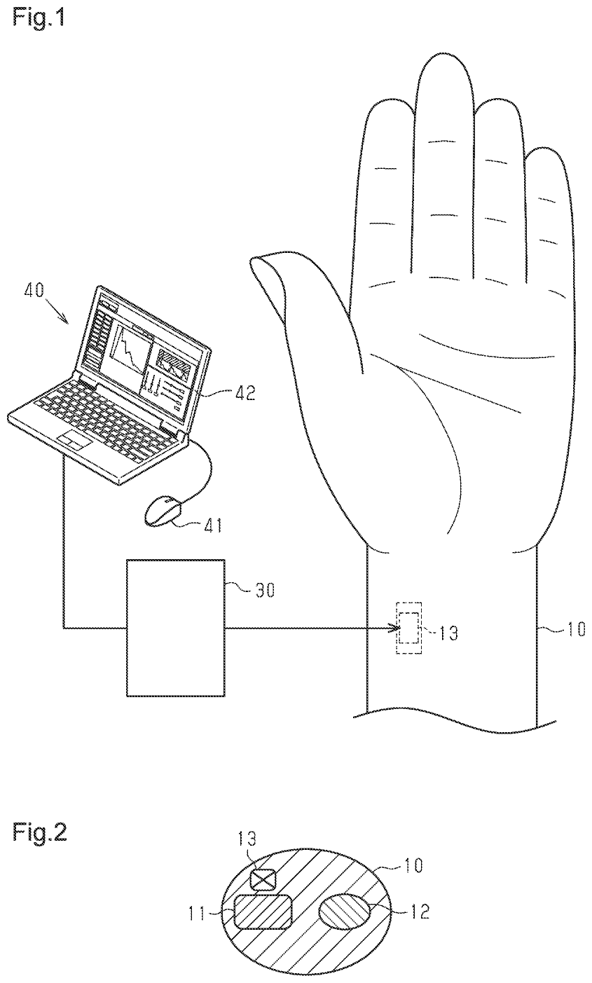

As shown in FIGS. 1 and 2, the pulsation generating apparatus includes a dummy 10. The dummy 10 imitates the outer shape of a front arm and a hand of a human body and is made of flexible material. Examples of the flexible material of the dummy 10 include elastomers, such as silicone or urethane.

The dummy 10 internally includes a first core 11, a second core 12, and a sheet-shaped dielectric elastomer actuator (DEA) 13. The first core 11 and the second core 12 imitate the radius and the ulna of a human body, respectively. The DEA 13 imitates a radial artery.

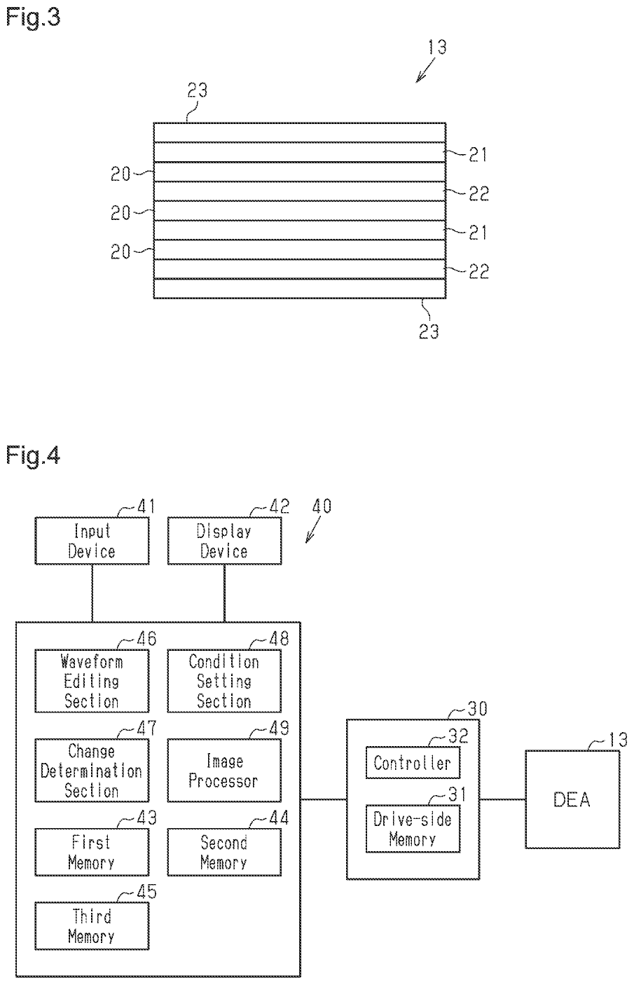

As shown in FIG. 3, the DEA 13 is a multi-layer structure formed by laminating sets of a dielectric layer 20, a positive electrode 21, and a negative electrode 22. The dielectric layer 20 is made of dielectric elastomer and has a sheet shape. The positive electrode 21 and the negative electrode 22 are electrode layers on the opposite sides of the dielectric layer 20 in the thickness direction. An insulating layer 23 is laminated on each of the outermost layers of the DEA 13. When a direct-current voltage is applied to a section between the positive electrode 21 and the negative electrode 22, the DEA 13 deforms in correspondence with the magnitude of the applied voltage such that the dielectric layer 20 is compressed in the thickness direction and extended in a surface direction of the DEA 13. The surface direction of the DEA 13 extends along the surface of each dielectric layer 20.

The dielectric elastomer of the dielectric layer 20 is not particularly limited and may be a dielectric elastomer used for a typical DEA. Examples of the dielectric elastomer include crosslinked polyrotaxane, silicone elastomer, and urethane elastomer. One of these types of dielectric elastomer may be used alone, or two or more of these may be used in combination. The thickness of the dielectric layer 20 is, for example, 20 to 200 μm.

Examples of the materials of the positive electrode 21 and the negative electrode 22 include conductive elastomer, carbon nanotube, Ketjenblackack®, and metal vapor deposition film. Examples of the conductive elastomer include a conductive elastomer that contains an insulating polymer and a conductive filler.

Examples of the insulating polymer include crosslinked polyrotaxane, silicone elastomer, and urethane elastomer. One of these types of insulating polymer may be used alone, or two or more of these may be used in combination. Examples of the conductive filler include Ketjenblack®, carbon black, and metal particle such as copper or silver. One of these types of conductive filler may be used alone, or two or more of these may be used in combination. The thickness of each of the positive electrode 21 and the negative electrode 22 is, for example, 0.01 to 100 μm.

The dielectric elastomer of the insulating layer 23 is not particularly limited and may be a dielectric elastomer used for an insulating portion of a typical DEA. Examples of the insulating elastomer include crosslinked polyrotaxane, silicone elastomer, acrylic elastomer, and urethane elastomer. One of these types of insulating elastomer may be used alone, or two or more of these may be used in combination. The thickness of the insulating layer 23 is, for example, 3 to 100 μm.

As shown in FIGS. 1 and 4, the pulsation generating apparatus includes a drive unit 30 and a waveform editing device 40. The drive unit 30 is configured to apply a cyclically-changing voltage to a section between two electrodes (the positive electrode 21 and the negative electrode 22 of the DEA 13). The waveform editing device 40 is configured to edit the waveform of a voltage applied to the DEA 13 by the drive unit 30. The drive unit 30 includes a drive-side memory 31 and a controller 32. The drive unit 30 may he circuitry including: 1) one or more processors that operate according to a computer program (software); 2) one or more dedicated hardware circuits such as application specific integrated circuits (ASICs) that execute at least part of various processes; or 3) a combination thereof. The processor includes a CPU and a memory such as a RAM and a ROM. The memory stores program codes or commands configured to cause the CPU to execute processes. The memory, or a computer readable medium, includes any type of media that are accessible by general-purpose computers and dedicated computers. The drive-side memory 31 stores drive waveform data that indicates changes in voltage corresponding to one cycle sent from the waveform editing device 40. The controller 32 repeatedly applies, from a power supply (not shown) to the DEA 13, a voltage having a waveform that is based on the drive waveform data stored in the drive-side memory 31.

The waveform editing device 40 is a computer that includes a pointing device 41 (input device), a display 42. a first memory 43, a second memory 44, a third memory 45, a waveform editing section 46, a change determination section 47, a condition changing section 48, and an image processor 49. That is, the waveform editing section 46, the change determination section 47, the condition changing section 48, and the image processor 49 may be circuitry including: 1) one or more processors that operate according to a computer program (software); 2) one or more dedicated hardware circuits such as application specific integrated circuits (ASICs) that execute at least part of various processes; or 3) a combination thereof. The processor includes a CPU and a memory such as a RAM and a ROM. The memory stores program codes or commands configured to cause the CPU to execute processes. The memory, or a computer readable medium, includes any type of media that are accessible by general-purpose computers and dedicated computers.

The pointing device 41 is, for example, a keyboard, a touch panel, or a mouse. The pointing device 41 receives, for example, an operation command from an operator. The display 42 is, for example, a display device such as a liquid crystal display or an organic EL display.

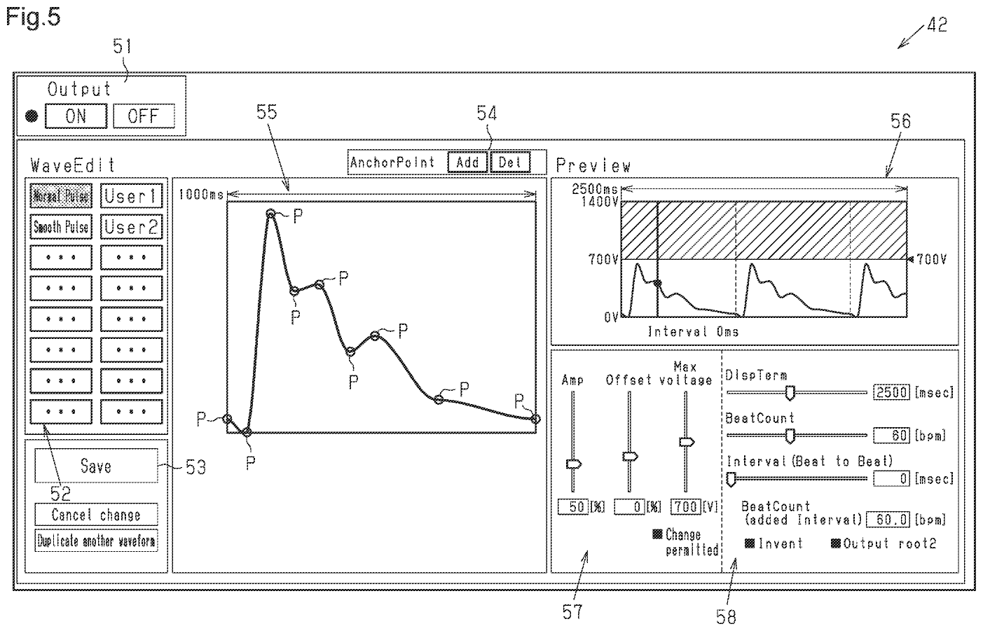

As shown in FIG. 5, the upper section of the display 42 displays an output button 51 used to switch the application of a voltage to the DEA 13 between on and off. The left section of the display 42 displays invocation buttons 52 used to invoke registered voltage waveform data and a save button 53 used to register new voltage waveform data.

The middle section of the display 42 displays anchor point buttons 54 that are respectively used to add and delete an anchor point P (described later) and a first waveform editing screen 55 that indicates a waveform subject to editing. The right upper section of the display 42 displays a driving situation screen 56 that indicates the voltage applied to the DEA 13. The middle lower section of the display 42 indicates a second waveform editing screen 57. The second waveform editing screen 57 includes a slider that changes the amplitude of an output (Amp), the magnitude of an offset voltage (Offset), and a maximum voltage (Max voltage). The right lower section of the display 42 displays a condition setting screen 58. The condition setting screen 58 includes a slider that sets a driving condition in the case of applying a voltage having a waveform based on the drive waveform data in the drive unit 30. Examples of the driving condition include the speed of one cycle (BeatCount) and the length of a standby time arranged between cycles (Interval).

The operator can operate the DEA 13 and edit the waveform of a voltage applied to the DEA 13 by operating the pointing device 41 so as to operate various buttons displayed on the display 42 and by changing the displayed contents of the first waveform editing screen 55 and the second waveform editing screen 57, Further, the operator can set the driving condition by operating the pointing device 41 so as to change the displayed content of the condition setting screen 58.

The first memory 43 stores registered voltage waveform data in association with the invocation buttons 52. The registered voltage waveform data includes voltage waveform data. corresponding to one cycle that reproduces a known pulsation pattern, such as normal pulse or smooth pulse, and voltage waveform data corresponding to one cycle created by the user. The normal pulse is a vibration pattern of artery in a case where a person is normal and healthy. The smooth pulse is a vibration pattern of an artery that occurs, for example, during pregnancy. The voltage waveform data that reproduces a known pulsation pattern is associated with the invocation button 52 that is named a pulse name such as “normal pulse.” The voltage waveform data created by the user is associated with the invocation button 52 that is named “User.”

The second memory 44 stores the drive waveform data that was sent to the drive unit 30 most recently (hereinafter referred to as the previous waveform data) and edit waveform data, which will be described below.

The third memory 45 stores programs that cause the waveform editing device 40 and the drive unit 30 to execute the processes of steps S13 to S13 and steps S21 to S24. The waveform editing device 40 and the drive unit 30 execute the processes of steps S11 to S13 and steps S21 to S24 in accordance with the programs.

The waveform editing section 46 creates edit waveform data corresponding to one cycle that is based on the previous waveform data sent to the drive unit 30, and causes the second memory 44 to store the edit waveform data. The edit waveform data stored in the second memory 44 is changed through an operation performed by the user. At a specific point in time, the waveform editing section 46 sends the current edit waveform data to the drive unit 30 as drive waveform data and updates the previous drive waveform data stored in the second memory 44.

The change determination section 47 refers to the comparison between the previous waveform data and the edit waveform data that are stored in the second memory 44 to determine whether the edit waveform data has been changed.

The condition changing section 48 sends, to the drive unit 30, the driving condition that has been set through an operation performed by the user.

The image processor 49 creates an image that indicates a waveform corresponding to edit waveform data and displays the image on the first waveform editing screen 55 of the display 42. The first waveform editing screen 55 displays a waveform that includes several anchor points P and Bezier curves. The anchor points P correspond to a start point, an end point, and inflection points of a waveform that corresponds to one cycle. Each of the Bezier curves connects adjacent ones of the anchor points P. The image processor 49 creates an image indicating a waveform in which the current driving condition is reflected on the previous drive waveform data stored in the second memory 44 and displays the image on the driving situation screen 56 of the display 42.

Various types of the voltage waveform data in the present embodiment include, as parameters that define a waveform, information related to the coordinates of all the anchor points P of a waveform corresponding to one cycle, the Bezier curves that each connect adjacent ones of the anchor points P, the magnitude of an output, the magnitude of an offset voltage, and the maximum voltage. The anchor point P corresponding to the start point and the anchor point P corresponding to the end point have the same value.

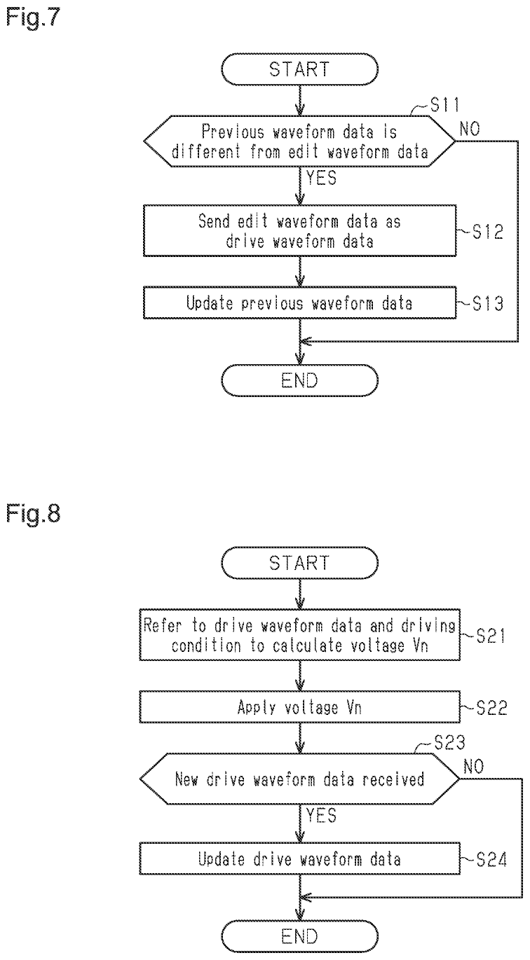

Referring to FIG. 7, while the DEA 13 is driven, the waveform editing device 40 repeatedly executes the following processes of steps S11 to S13 in a cycle of several milliseconds to several tens of milliseconds.

In step S11, the change determination section 47 compares the previous waveform data with the edit waveform data stored in the second memory 44 to determine whether the previous waveform data is different from the current edit waveform data. The determination of the difference between the previous waveform data and the current edit waveform data is based on whether the parameters included in the previous waveform data all match the parameters included in the current edit waveform data.

When determining in step S11 that the previous waveform data. is different from the current edit waveform data (YES), the waveform editing section 46 executes step S12 to send the current edit waveform data to the drive unit 30 as the drive waveform data. Next, in step S13, the waveform editing section 46 updates the previous waveform data stored in the second memory 44 and then ends the process. When determining in step S11 that the previous waveform data is not different from the current edit waveform data (NO), the change determination section 47 ends the process.

Referring to FIG. 8, while the DEA 13 is driven, the controller 32 of the drive unit 30 repeatedly executes the following processes of steps S21 to S24 in a cycle of several milliseconds to several tens of milliseconds.

In step S21, the controller 32 refers to the drive waveform data stored in the drive-side memory 31 and the driving condition received from the condition changing section 48 of the waveform editing device 40 to calculate a voltage Vn that should be applied next. Next, in step S22, the controller 32 applies the calculated voltage Vn to the DEA 13.

Subsequently, in step S23, the controller 32 determines whether new drive waveform data has been received from the waveform editing section 46 of the waveform editing device 40. When determining in step S23 that new drive waveform data has been received (YES), the controller 32 executes step S24 to update the drive waveform data stored in the drive-side memory 31 to the received new drive waveform data. Then, the controller 32 ends the process. When determining in step S23 that new drive waveform data has not been received (NO), the controller 32 ends the process.

Description will now be made for a method in which the pulsation generating apparatus of the present embodiment is used to generate a voltage waveform that operates the DEA 13 so as to present a particular tactile sense. The following description provides an example in which registered voltage waveform data corresponding to a normal pulse is edited to generate a voltage waveform that operates the DEA 13 so as to present a tactile sense that is closer to the pulsation pattern of a normal pulse felt by a skilled person during actual palpation.

First, a preparatory step is executed to drive the DEA 13 so as to operate the dummy 10 in a pulsation pattern that is based on registered voltage waveform data corresponding to a normal pulse.

More specifically, in a state in which the output button 51 is turned off, that is, when voltage is not applied to the DEA 13 from the drive unit 30 and the DEA 13 is deactivated, the pointing device 41 is operated to click the invocation button 52 corresponding to a normal pulse. In the waveform editing device 40, this operation causes the registered voltage waveform data corresponding to a normal pulse to be sent to the drive unit 30 as drive waveform data and updates the previous waveform data stored in the second memory 44. In the drive unit 30, the drive waveform data stored in the drive-side memory 31 is updated using the drive waveform data sent from the waveform editing device 40.

The waveform editing section 46 of the waveform editing device 40 creates new edit waveform data in which the registered voltage waveform data corresponding to a normal pulse is duplicated and updates the edit waveform data stored in the second memory 44. The image processor 49 creates an image that indicates a waveform corresponding to the edit waveform data and displays the image on the first waveform editing screen 55 of the display 42. The position of each slider displayed on the second waveform editing screen 57 is adjusted to the value of the registered voltage waveform data corresponding to a normal pulse.

Then, after the pointing device 41 is operated to turn the output button 51 on, the processes shown in FIG. 8 are repeatedly executed in the drive unit 30. This causes the voltage that changes in correspondence with the drive waveform data stored in the drive-side memory 31 (i.e., the registered voltage waveform data corresponding to a normal pulse) to be applied to the DEA 13 so as to operate the DEA 13. Further, the processes shown in FIG. 7 are repeatedly executed in the waveform editing device 40.

Next, an editing step is executed to edit a waveform. In the editing step, the user is a skilled person at palpation. In the editing step, the user uses one hand to touch the dummy 10 so as to experience the pulsation presented from the dummy 10 in correspondence with the operation of the DEA 13 while the user uses the other hand to operate the pointing device 41 so as to edit the edit waveform data.

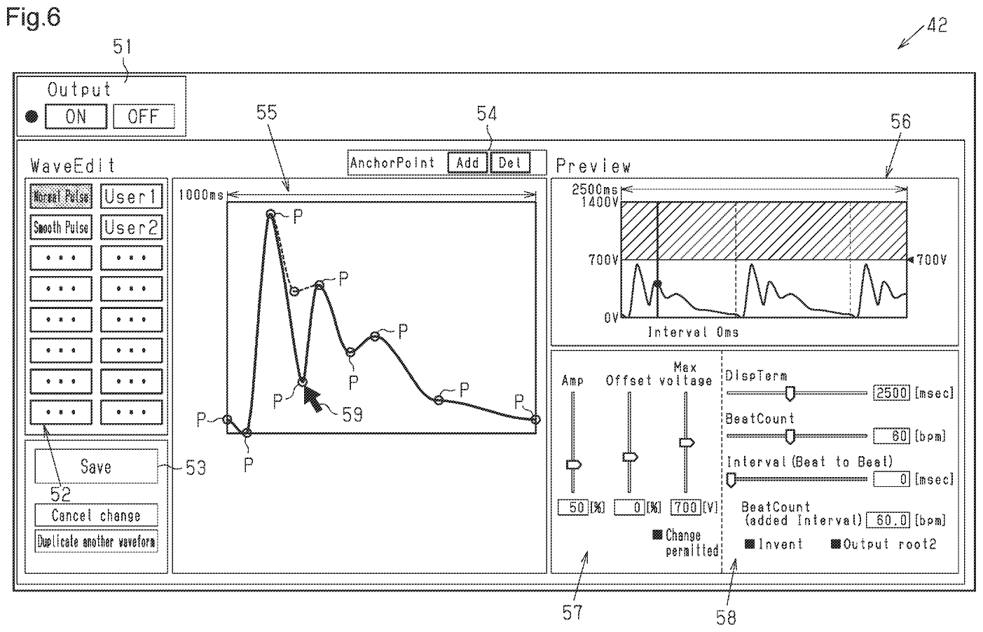

Referring to FIG. 6, the user changes the waveform displayed on the first waveform editing screen 55 by moving the anchor points P onto the image of the waveform displayed on the first waveform editing screen 55 and by adding or deleting the anchor points P. The anchor points P are moved through a general operation using a pointing device, for example, by moving a pointer 59 displayed on the first waveform editing screen 55 onto a target anchor point P (i.e., performing drag and drop) or by operating an arrow key on the keyboard with the target anchor point P selected. The sections between the anchor points P are automatically complemented by the Bezier curves. Further, the user operates the pointing device 41 to change the position of each slider displayed on the second waveform editing screen 57. For example, to return to the registered voltage waveform data corresponding to a normal pulse or to use another registered voltage waveform data as a basis, the invocation button 52 corresponding to target registered voltage waveform data is clicked so as to change the waveform displayed on the first waveform editing screen 55.

When the user performs an operation to change the displayed contents of the first waveform editing screen 55 and the second waveform editing screen 57, the waveform editing section 46 changes the edit waveform data stored in the second memory 44 to edit waveform data that is based on the displayed contents of the first waveform editing screen 55 and the second waveform editing screen 57.

As shown in the flowchart of FIG. 7, while the DEA 13 is driven, the waveform editing device 40 performs step S11 to regularly execute the process that determines whether the previous waveform data stored in the second memory 44 is different from the edit waveform data. Thus, when the edit waveform data is changed, the previous waveform data is determined as being different from the current edit waveform data in step S11 of the current or next cycle. Then, in step S12, the current edit waveform data is sent to the drive unit 30 as the drive waveform data.

As shown in the flowchart of FIG. 8, while the DEA 13 is driven, the drive unit 30 performs step S23 to regularly execute the process that determines whether new drive waveform data has been received from the waveform editing section 46. Thus, after new drive waveform data is received, the new drive waveform data is determined as having been received in step S23 of the current or next cycle. In step S24, the drive waveform data stored in the drive-side memory 31 is updated to the received new drive waveform data. Then, in steps S21 and S22 of the next cycle or the cycle after the next, the received new drive waveform data is used to calculate the voltage Vn that should be applied next and apply the calculated voltage Vn to the DEA 13. This changes the operation of the DEA 13 to an operation that is based on the received new drive waveform data (i.e., the edit waveform data that has been edited by the user).

The processes shown in FIGS. 7 and 8 are repeatedly executed in a cycle of several milliseconds to several tens of milliseconds. Thus, immediately after the user performs an operation to change the displayed contents of the first waveform editing screen 55 and the second waveform editing screen 57, the operation of the DEA 13 is changed to an operation based on the changed edit waveform data. This changes the vibration pattern transmitted to the hand of the user that touches the dummy 10.

As shown in the flowchart of FIG. 7, after the current edit waveform data is sent to the drive unit 30 as the drive waveform data in step S12, the previous waveform data stored in the second memory 44 is updated in step S13. Then, in step S11 of the next cycle, the determination based on the updated previous waveform data is executed.

For the vibration pattern of the pulsation transmitted from the dummy 10 to one hand of the user to become close to the pulsation pattern of a normal pulse based on his or her experience, the user operates the other hand to operate the pointing device 41 so as to change the displayed contents of the first waveform editing screen 55 and the second waveform editing screen 57 and edit the edit waveform data. This changes the vibration pattern of the pulsation transmitted from the dummy 10 every time the edit waveform data is edited. When the vibration pattern transmitted from the dummy 10 matches the vibration pattern of the normal pulse based on his or her experience, the user clicks the save button 53 to update the registered voltage waveform data corresponding to a normal pulse to the current edit waveform data or register the current edit waveform data as new voltage waveform data created by the user. This provides a voltage waveform that operates the DEA 13 so as to present a tactile sense that is closer to the pulsation pattern of a normal pulse felt by a skilled person during actual palpation.

The advantages of the present embodiment will now be described.

(1) The pulsation generating apparatus includes the DEA 13, the drive unit 30, and the waveform editing section 46. The DEA 13 includes two electrodes. The drive unit 30 is configured to drive the DEA 13 by repeatedly applying, to a section between the two electrodes, a voltage that changes in correspondence with the drive waveform data that indicates voltage changes corresponding to one cycle. The waveform editing section 46 is configured to change the edit waveform data in correspondence with an operation performed by the user. When the edit waveform data is changed during driving of the DEA 13, the pulsation generating apparatus is configured to update the drive waveform data. such that the changed edit waveform data becomes new drive waveform data and drive the DEA 13 using the updated drive waveform data.

In the configuration, when the edit waveform data is changed during driving of the DEA 13, the operation of the DEA 13 is switched to an operation that is based on the changed edit waveform data without performing an operation such as saving or sending the changed edit waveform data. This allows the user to smoothly search for a voltage waveform that produces a particular motion of the DEA 13 while editing the edit waveform data. As a result, the voltage waveform is generated efficiently.

(2) The pulsation generating apparatus includes the change determination section 47 configured to regularly determine whether the edit waveform data has been changed. The pulsation generating apparatus is configured to update the drive waveform data when the change determination section 47 determines that the edit waveform data has been changed.

The configuration reduces the frequency of updating the drive waveform data used to drive the DEA 13.

(3) The pulsation generating apparatus includes the image processor 49 configured to cause the display 42 to display an image that corresponds to the edit waveform data. The waveform editing section 46 is configured to change the edit waveform data in correspondence with the user's operation that changes the image displayed on the display 42 using the pointing device 41.

The configuration allows the user to edit a waveform intuitively. Thus, even a user who has a small amount of knowledge related to machine or information processing easily generates a voltage waveform that produces a particular motion of the DEA 13.

(4) When an operation is performed to invoke registered voltage waveform data during driving of the DEA 13, the edit waveform data is changed to the registered voltage waveform data.

The configuration easily changes the operation of the DEA 13 during driving to an operation that is based on the registered voltage waveform data.

The present embodiment may he modified as follows. The present embodiment and the following modifications can be combined as long as they remain technically consistent with each other.

The parameters that define waveforms included in various types of voltage waveform data are not limited to the parameters of the above-described embodiment. For example, some of the parameters of the above-described embodiment may be omitted. Alternatively, another parameter may be added.

The processes of steps S23 and S24 in the processes shown in the flowchart of FIG. 8, that is, the processes that determine whether new drive waveform data has been received from the waveform editing section 46 and update the drive waveform data in a case where the new drive waveform data has been received, do not have to be executed every time. In other words, the frequency and the point in time at which the processes of steps S23 and S24 may be changed. For example, the processes of steps S23 and S24 are executed at a specific point in time that is the end of changes in voltage corresponding to one cycle based on the drive waveform data, and the processes of steps S23 and S24 are not executed at other points in time. in this case, the processes of steps S23 and S24 are executed only one time while the voltage corresponding to one cycle based on the drive waveform data is changing.

The change determination section 47 may be omitted. In this case, the process of step S11 in the flowchart of FIG. 7 is omitted so that the processes of steps S12 and S13 are executed every time, and the process of step S23 in the flowchart of FIG. 8 is omitted so that the process of S24 is executed every time.

The pointing device 41 and the display 42 may be external devices that are prepared separately from the actuator device of the present disclosure.

The number of the DEAs 13 arranged in the dummy 10 is not particularly limited.

The DEAs 13 may be replaced with other electroactive polymer actuators (EPA) such as ionic polymer metal composites (IPMC).

The actuator device of the present disclosure may be employed as a tactile sense presentation device other than the pulsation generating apparatus that causes the user to recognize, as a tactile sense, an operation such as vibration generated in correspondence with applied voltage. The actuator device of the present disclosure can be employed not only in the tactile sense presentation device but also in most types of devices that change applied voltage so as to produce a particular motion in electroactive polymer actuator.

Some or all of the electroactive polymer actuator such as the DEA 13, the drive unit 30, and the waveform editing device 40, which form the actuator device, may be integrally formed. For example, the electroactive polymer actuator may be formed integrally with the drive unit 30. Alternatively, the drive unit 30 may be formed integrally with the waveform editing device 40. Instead, the electroactive polymer actuator, the drive unit 30, and the waveform editing device 40 may be integrally formed.

The programs that cause the actuator device to execute the processes of steps S11 to S13 and steps S21 to S24 may be stored in a memory device incorporated in the actuator device or may be stored in an external memory device such as removable media. The programs may be stored in a WEB server and executed in the WEB server.

### DESCRIPTION OF THE REFERENCE NUMERALS

P) Anchor Point; 10) Dummy; 13) Dielectric Elastomer Actuator (DEA); 30) Drive Unit; 31) Drive-side Memory; 32) Controller; 40) Waveform Editing Device; 41) Pointing Device; 42) Display; 43) First Memory; 44) Second Memory; 45) Third Memory; 46) Waveform Editing Section; 47) Change Determination Section; 48) Condition Changing Section; 49) Image Processor; 55) First Waveform Editing Screen; 56) Second Waveform Editing Screen

1. An actuator device, comprising:
an electroactive polymer actuator that includes two electrodes;
a drive unit configured to drive the electroactive polymer actuator by repeatedly applying, to a section between the two electrodes, a voltage that changes in correspondence with drive waveform data that indicates voltage changes corresponding to one cycle; and
a waveform editing section configured to change edit waveform data in correspondence with an operation performed by a user,
wherein when the edit waveform data is changed during driving of the electroactive polymer actuator, the actuator device is configured to update the drive waveform data such that the changed edit waveform data becomes new drive waveform data and drive the electroactive polymer actuator using the updated drive waveform data.

2. The actuator device according to claim 1, comprising a change determination section configured to regularly determine whether the edit waveform data has been changed,
wherein the actuator device is configured to update the drive waveform data when the change determination section determines that the edit waveform data has been changed.

3. The actuator device according to claim 1, comprising an image processor configured to cause a display to display an image that corresponds to the edit waveform data,
wherein the waveform editing section is configured to change the edit waveform data through an operation performed by the user, the operation changing the image displayed on the display using a pointing device.

4. The actuator device according to claim 1, wherein the actuator device is employed as a tactile sense presentation device that causes the user to recognize, as a tactile sense, an operation that is based on expansion and contraction of the electroactive polymer actuator.

5. The actuator device according to claim 4, wherein the tactile sense presentation device is a pulsation generating apparatus that causes the user to recognize, as a tactile sense of pulsation, vibration that is based on the expansion and contraction of the electroactive polymer actuator.

6. The actuator device according to claim 1, wherein when an operation is performed to invoke voltage waveform data stored in advance during driving of the electroactive polymer actuator, the actuator device is configured to change the edit waveform data to the voltage waveform data.

7. A method for generating a voltage waveform, the method comprising producing a waveform of applied voltage that produces a particular motion of the electroactive polymer actuator using the actuator device according to claim 1.

8. A method for driving an electroactive polymer actuator, the method comprising:
driving the electroactive polymer actuator by repeatedly applying a voltage that changes in correspondence with drive waveform data that indicates voltage changes corresponding to one cycle;
changing edit waveform data in correspondence with an operation performed by a user; and
when the edit waveform data is changed during driving of the electroactive polymer actuator, updating the drive waveform data such that the changed edit waveform data becomes new drive waveform data,
wherein the driving includes driving the electroactive polymer actuator using the drive waveform data. updated in the updating.

9. A non-transitory computer-readable medium storing a program that controls an actuator device, the actuator device including:
an electroactive polymer actuator that includes two electrodes;
a drive unit configured to drive the electroactive polymer actuator by repeatedly applying, to a section between the two electrodes, a voltage that changes in correspondence with drive waveform data that indicates voltage changes corresponding to one cycle; and
a waveform editing section configured to change edit waveform data in correspondence with an operation performed by a user,
wherein when the edit waveform data is changed during driving of the electroactive polymer actuator, the program causes the actuator device to execute a process that updates the drive waveform data such that the changed edit waveform data becomes new drive waveform data and drives the electroactive polymer actuator using the updated drive waveform data.
